(19) United States
(12) Patent Application Publication
(10) Pub. No.: US 2002/0098509 A1
(43) Pub. Date: Jul. 25, 2002

(54) Method for the quantitative determination of reversible acting inhibitors of the oxidoreductases

(76) Inventor: Heike Jurgens, Unterluss (DE)

Correspondence Address: Norris McLaughlin & Marcus, 30th Floor, 220 East 42nd Street, New York, NY, 10017, US

(21) Appl. No.: 10/099,328

(22) Filed: Mar. 14, 2002

Related U.S. Application Data
(63) Continuation of application No. 08/765,114, filed on Dec. 10, 1996.
Application No. 08/765,114 is a 371 of international application No. PCT/EP96/01573, filed on Apr. 15, 1996.

(30) Foreign Application Priority Data
Apr. 13, 1995 (DE) 195 14 004.4

(51) Int. Cl. C12Q 1/68

(52) U.S. Cl. 435/6

(57) ABSTRACT

A method for the quantitative determination of a reversible inhibitor of an oxidoreductase by reacting an oxidoreductase, for example, sarcosine oxidase, and a substrate therefore, for example, sarcosine, in the presence of an unknown concentration of a reversible inhibitor of said reaction, then measuring the enzyme activity of said oxidoreductase, and then using the measurement of enzyme activity to determine the concentration of said reversible inhibitor. Also disclosed is a measuring device for carrying out the method. The device is composed of a reaction chamber, at least two piston pumps, which are each connected to the reaction chamber by feeding tubes, and a detector, which is inside the reaction chamber or following the reaction chamber and operably connected thereto. Also, disclosed are test kits for use in carrying out the method.

In food, in pharmaceutical and in chemical industry in general, the collection of data is of immense importance for substance characterization and for process control. The same applies to environmental technology as well as to water, wastewater and soil monitoring.

Commonly used selective methods for the determination of those data and controlled variables are chromatographic techniques like high pressure liquid chromatography (HPLC), gas chromatography (GC), wet analytical methods such as volumetric analytical methods and enzyme techniques which commonly are test kits. The disadvantage of the HPLC technique is its long time to get the data and the sample treatment. Also the cost for consumption material is quite high and finaly there's a large instrumental expenditure necessary. The same points are also valid for the GC technique. The wet analysis techniques also take a lot of time to get the analytical data, there is quite an expense in sample treatment, and a great amount of sample necessary, although there is only low selectivity. When using test kits, duration of a single analysis is also quite long. It is also important to state the fact, that test kits on the market are only available for a small amount of analytes.

Those analytes are for instance the so called reversible inhibitors. They include substances like carboxylic acids, amino acids and their salts, formaldehyde, acetaldehyde, n-methyl-amino acids, methoxyacetate, pyrrole-2-carboxylic acid, thiophene-2-carboxylic acid, furan-2-carboxylic acid.

Examples for oxidoreductases are sarcosine oxidase, monoamino oxidase, L-lactate 2-monooxygenase and L-amino acid oxidase.

Thus, it is an object of this invention, to provide an analytical method to determine such data and controlled variables, which delivers short time relyable results on the concentration of the analyte, without immense apparative expenses.

Furthermore, it is the aim of this invention to deliver a measuring device to carry out such an analytical method.

According to a first aspect this aim is solved by a method for the quantitative determination of reversible working inhibitors of enzymatic reactions, in which an enzyme substrate is supplied and in presence of the inhibitor the enzyme activity of the enzyme, which is involved in the enzymatic reaction, is determined and from this resulting data, the concentration of the reversive acting inhibitor is calculated.

In the inventional method the analyte is the inhibitor. In the present invention, the inhibitor, which could lead to wrong data in conventional analytical systems, is used as data determined variable here. The influence of the inhibitor on the measurement is therefore used for the measurement of the inhibitor concentration, where the enzyme substrate is not, like in conventional methods the analyte, but the competitive reaction partner, which, together with the inhibitor, has to be present in the reaction mixture. The reaction participants, which are formed or consumed by the catalytic reaktion of the enzyme, are then detected by a conventional method. There is the possibility of electrochemical, amperometric or optical, for instance photometric, detection.

In the following the invention is illustrated by a reaction system, using the enzyme sarcosine oxidase (SOD) and sarcosine as a possible substrate. It is also possible to use n-methyl-L-leucine, n-methyl-DL-alanine, n-methyl-DL-valine, n-ethyl-glycine or heterocyclic amines as a substrate of the sarcosine oxidase.

In order to carry out the reaction, the enzyme can be present in solution or immobilized on a solid phase. The reaction system can be described by the following equation:

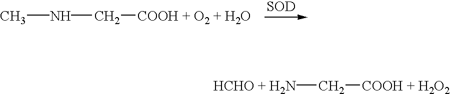

In this system, for example, reversible acting inhibitors are acetic acid, propionic acid and their salts, as well as formaldehyde, acetaldehyde, n-methyl-amino acid, methoxyacetate, pyrrol-2-carboxylic acid, thiophene-2-carboxylic acid and furan-2-carboxylic acid. By addition of these analytes to the reaction system, oxygen consumption and hydrogen peroxide production are being diminshed.

The advantage of the reaction system is its high selectivity. Matrix effects, which are produced by interfering substances, can be determined by variation of substrate and enzyme concentration as well as buffer composition. Furthermore, selectivity can be influenced by adding reversible acting inhibitors to the reaction system.

A loss in enzyme activity of the usually quite stable sarcosine oxidase can be compensated by increasing substrate concentration. A regeneration of the enzyme is not necessary, because the inhibiting substances are working reversible. Data aquisition is done without any further intermediate stages directly by means of an oxygen or hydrogen peroxide detection.

The necessary reagents to carry out the determination can be collected in a so called test kit. For instance, it could consist of a substrate of the sarcosine oxidase, sarcosine oxidase, e.g. in immobilized state, a buffer, e.g. phosphate buffer and stabelizers like EDTA.

A possible measuring device to perform the invented method consists of at least two piston pumps, which are connected to a reaction chamber via tubing. Preferable valves are installed in between the tubing, which allows to cut off, by-pass or supply the necessary reaction solutions. Preferably, the piston pumps are installed for sample, buffer and if necessary further reagent. A detector unit can be placed either in the reaction chamber itself, or following it. Principly, the kind of detection system used is not important. For instance, detection can be done by a photometric system or by measuring the decrease of oxygen or the formation of hydrogen peroxide, respectively. In case of an enzymatic reaction, an enzyme can be immobilized inside the reaction chamber or likewise for determination brought into it as well. The stirring of the reaction mixture inside the chamber can be performed by a magnetic stirrer or other mixing devices. An output can be connected to the reaction chamber. Preferably the piston pump can be driven by a motor. Piston and cylinder of the piston pump are made from plastics, metall or glass.

To perform a measurement the piston pumps are handled simultanously or in an appropriate time sequence, resulting in a parallel flow of the reaction partners along the way to the reaction chamber. After sufficient mixing inside the reaction chamber the measurement is done. In this manner, discontinous or continous processes can be carried out.

In the following the invented method is described according to the invention by means of flow injection analysis, stirred vessel and parallel flow analysis systems.

Flow Injection Analysis Systems (FIA Systems)

In FIA systems an injected volume of the sample is transported to the detector by a continuous moving, not segmented carrier or transport flow via a mixing and reaction line. Meanwhile, the injected sample zone is broadened (dispersion) and reacts with the ingredients of the carrier flow. As a result of the chemical conversion, the changes in concentration of the participating components lead to a change of signal at the detector, which is characteristic for the concentration of the determined substance of the injected sample.

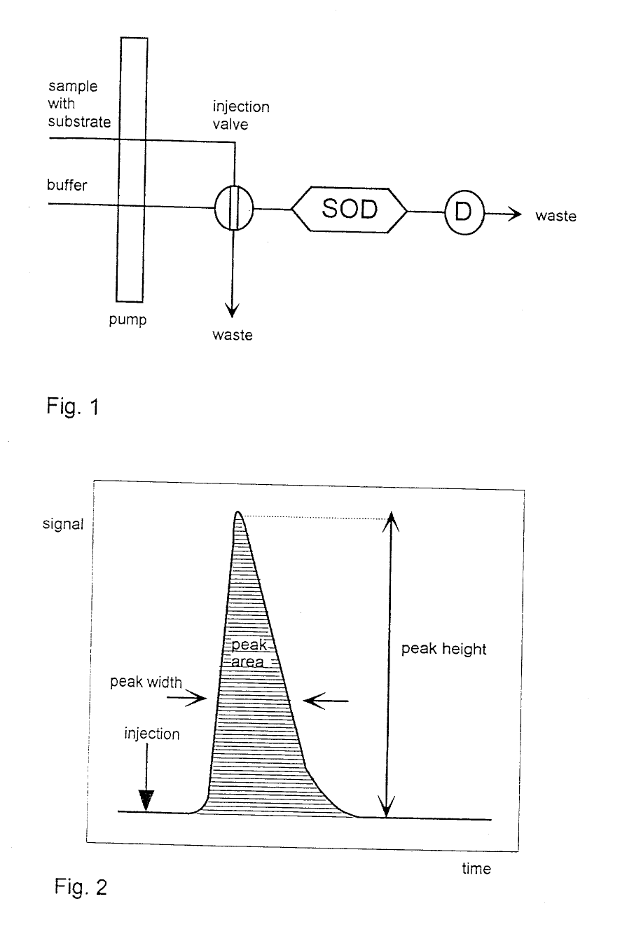

In FIG. 1 the analyte (acetic acid) as well as the substrate (sarcosine) are injected as a sample into the carrier flow consisting of the working buffer. The mixing and reacting line contains the immobilized enzyme sarcosine oxidase, which reacts with the analyte and the substrate of the sample. The decrease of oxygen or the production of hydrogene peroxide is measured by means of an oxygen or hydrogene peroxide electrode.

A typical measuring signal is reproduced in FIG. 2. The peak high H or surface S is proportional to the concentration of the sample.

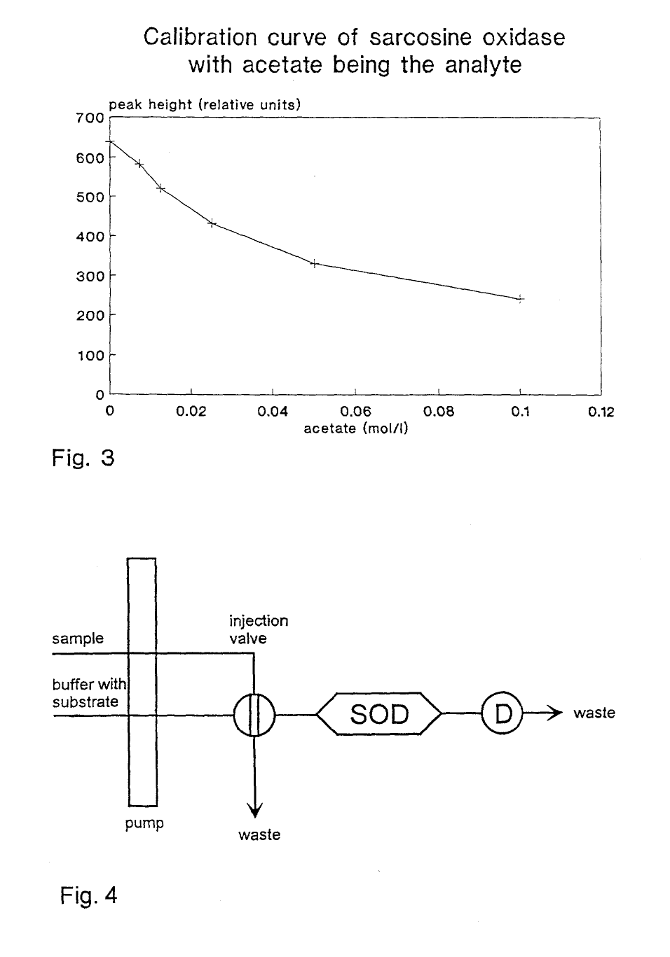

When calibrating via the peak heights, samples of different analyte concentrations are injected and the resulting peak heights in dependence of their analyte concentrations are drawn into a chart (FIG. 3). The sample of unknown analyte concentration is injected in the same manner and can be determined via the peak height with the help of the calibration curve.

As a result of the separation of enzyme and reaction media the immobilized enzyme, which still has it's catalytic activity, is kept in the system, while permanently new educts are added and products are removed.

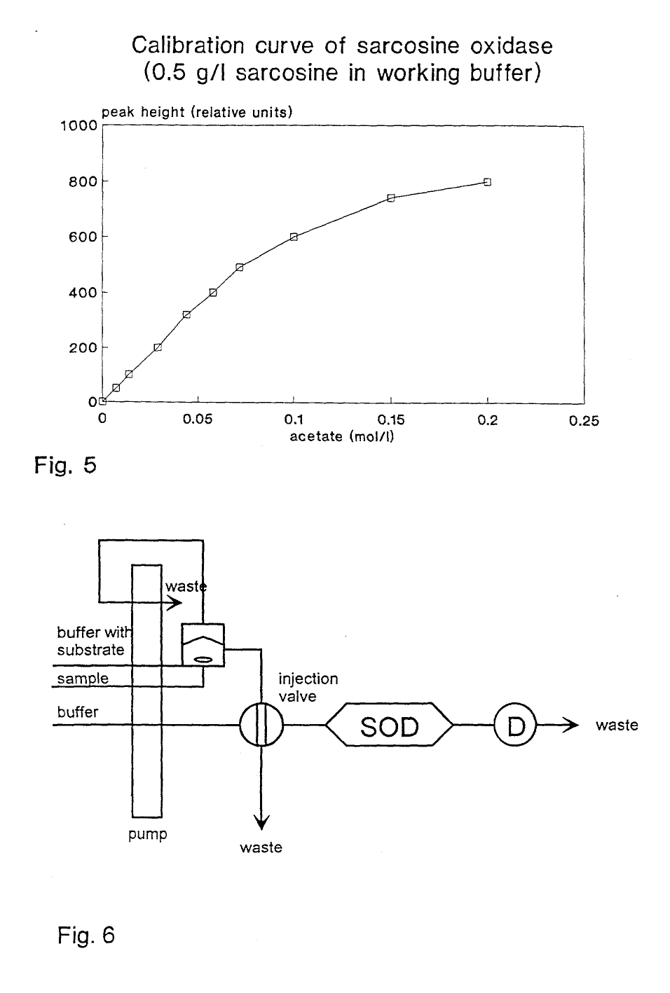

In FIG. 4 the substrate is added to the buffer solution and calibration is done by injecting the analyte. Again the analysis of the unknown sample can be be done by using the calibration curve as described in FIG. 5.

FIG. 6 shows the automatical supply of analyte as well as substrate via the mixing unit.

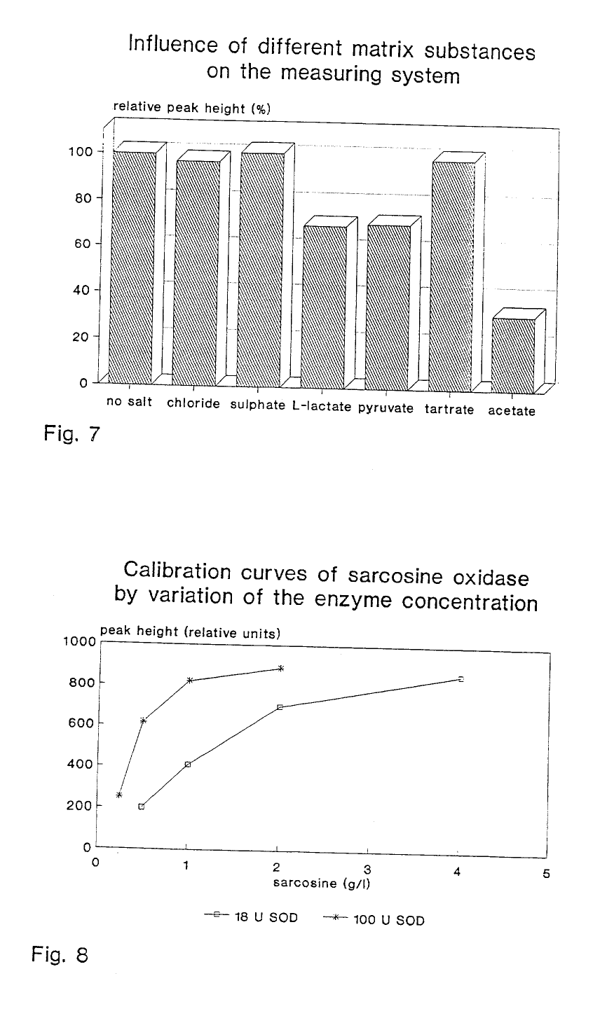

In FIG. 7 the different amount of inhibitor strength of substances, which often arise in biotechnological media, is shown. A maximum concentration of 0.1 mol/l is chosen for such kind of media. It is easy to see that the measuring system is inhibited by acetate quite strong but only very slight by the other substances.

FIG. 8 shows two curves of different sarcosine oxidase activities, whereby 18 U and 100 U sarcosine oxidase with rising substrate concentration were used.

It can be learned that the curves show a difference in linearity of the measuring scale. At a sarcosine oxidase activity of 18 U the linear measuring scale extends up to 2 g/l at low sensitivity, while using an activity of 100 U a linear measuring scale is received up to 0.5 g/l sarcosine at high sensitivity.

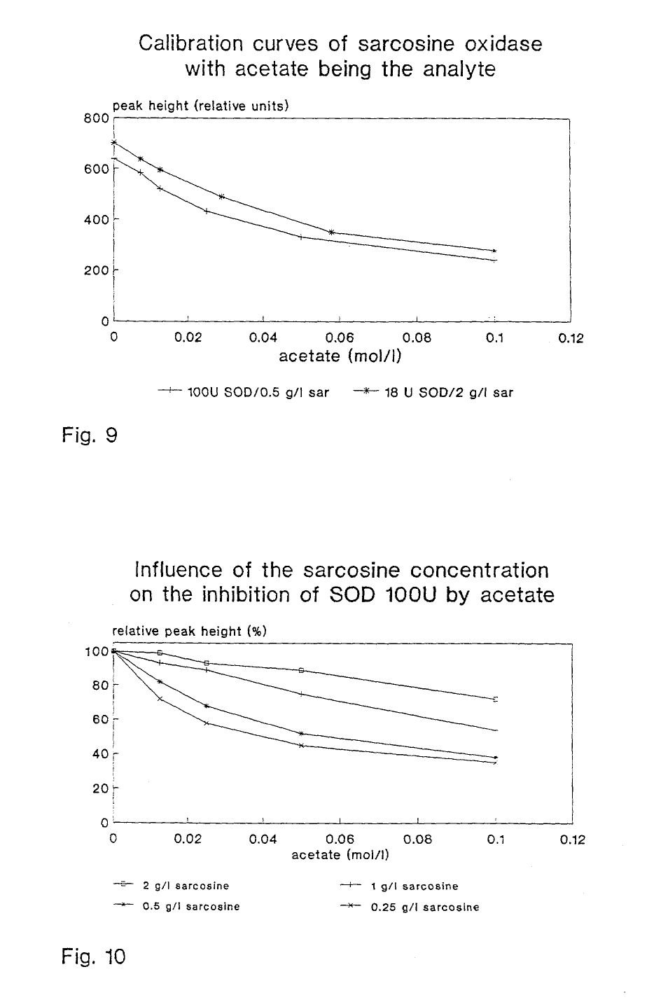

The calibration curves using acetate as analyte and their corresponding different enzyme activities are shown in FIG. 9. Substrate concentration for a sarcosine oxidase activity of 100 U is at 0.5 g/l sarcosine, while at the lower sarcosine oxidase activity 2 g/l sarcosine has to be used to receive nearly the same peak height. The measurement shows the fact that a possible decrease of enzyme activity can be compensated by increasing the substrate concentration and therefore maintain the course of the diagram.

A supposition for it is to chose substrate concentrations at both enzyme activities which are within the linear scale shown in FIG. 8.

FIG. 10 displays the influence of four different sarcosine concentrations onto the inhibition of the sarcosine oxidase activity (100 U) in relation to the acetate concentration. It is clear that at 0.25 g/l respectively 0.5 g/l sarcosine a considerable amount of inhibition takes place, whereas especially at low acetate concentrations up to 0.02 mol/l a high sensitivity is given. On the other hand at concentrations of 1 g/l or 2 g/l sarcosine only a small amount of inhibition can be observed, althought it covers a wide linear scale of the acetate concentration.

The measurements show, that by choosing of the right substrate concentration the amount of inhibition can be varied, resulting in a flexible measuring system which can be easily adjusted to the specific measuring problem.

Stirred Vessel System

In contrast to the FIA system the stirred vessel system is a discontinuous process. In order to perform a measurement all necessary reaction partners are brought into the reaction chamber of the stirred vessel system and the course of reaction is been monitored by the appropriate detectors during a fixed time period. Afterwards the whole reaction mixture is removed. Then a new measurement can be done.

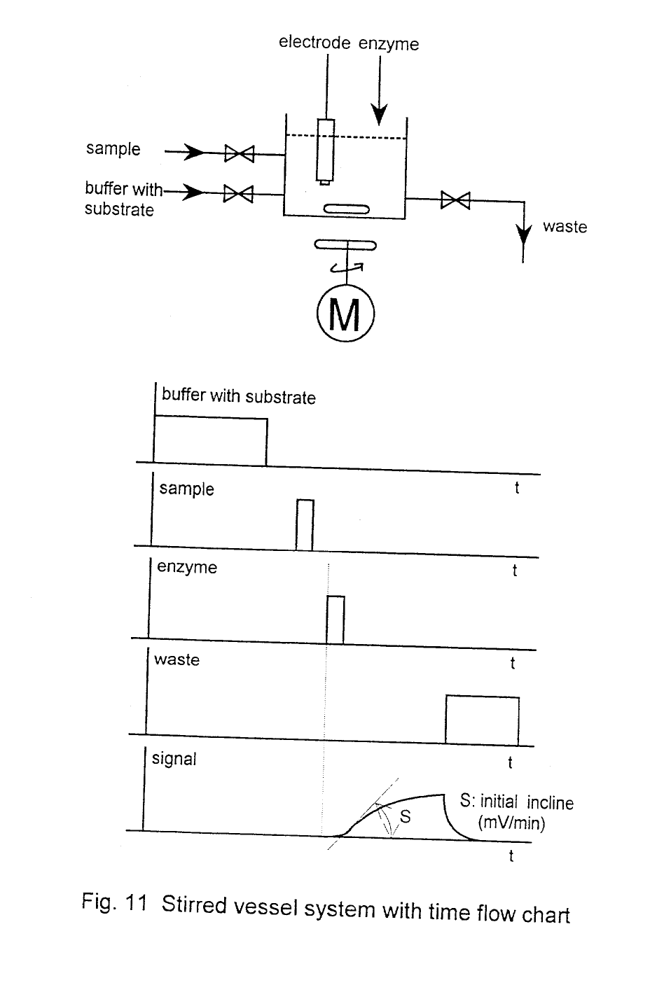

The reaction mixture of the stirred vessel system in FIG. 11 consists of the sample, substrate, buffer solution and enzyme. Here, the sample and the enzyme are added one after another and the sample itself containing the analyte, acetic acid for instance, in different concentrations. The decrease of oxygen or the production of hydrogene peroxide is measured by means of an oxygen or hydrogen peroxide sensitive electrode in a time resolved manner. The result of the measurement is calculated from the initial gradient of the signal.

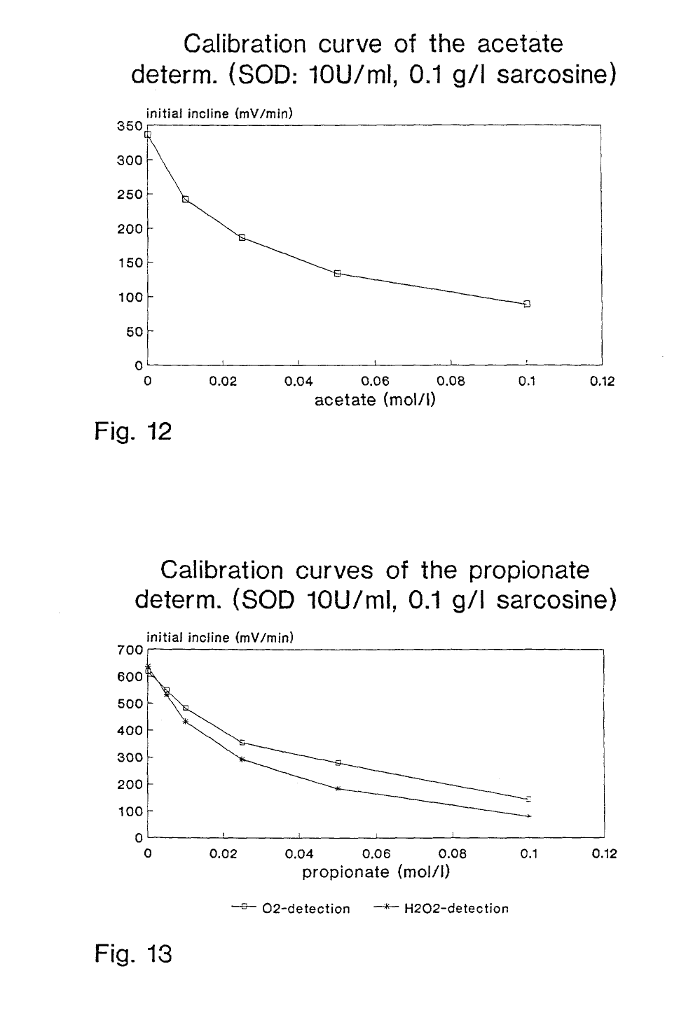

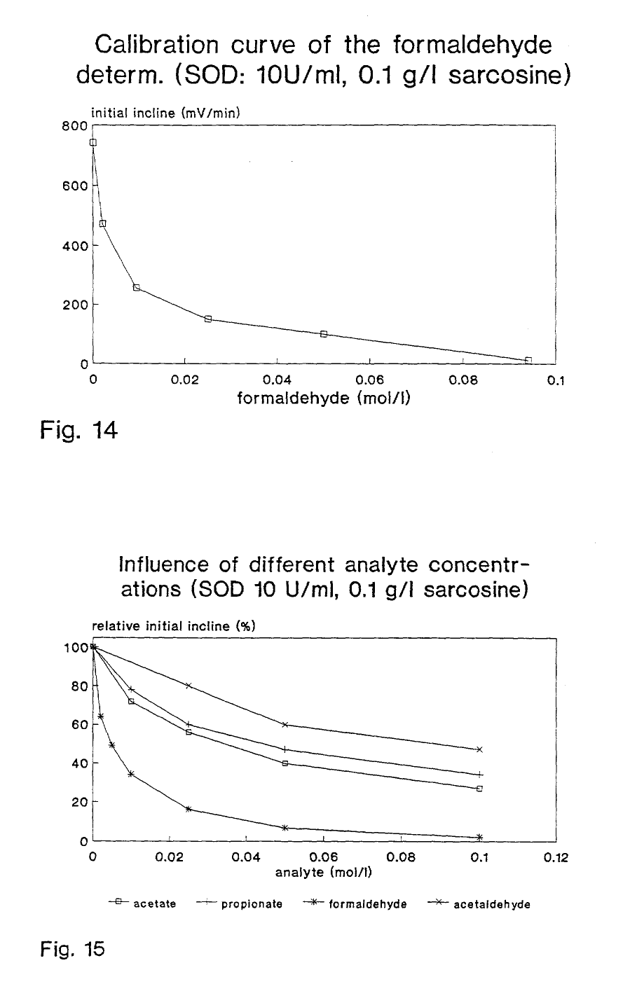

During the calibration via the initial gradient samples of different analyte concentration are added and the relating initial inclines are drawn into the chart in dependence of the analyte concentration (see FIGS. 12, 13 and 14). The sample containing the unknown analyte concentration is added to the reaction mixture in the same manner and is determined by using the initial incline which is compared to the calibration curve.

In FIG. 13 the calibration curves for the measurement of propionate with the simultaneous detection of oxygen and hydrogen peroxide are shown.

In FIG. 15 the relative initial incline in dependence of the respective analyte concentration is shown. It is evident that acetate and propionate are resulting in similar curves, while acetaldehyde is resulting in a lower and formaldehyde in a substantial higher amount of inhibition of the sarcosine oxidase. The highest sensitivity of those analytes can be found in the lower concentration area.

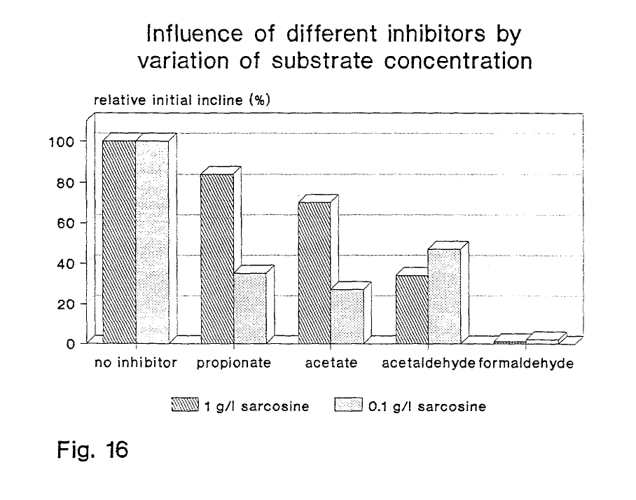

FIG. 16 shows the effect of inhibitors like acetate, propionate, acetaldehyde and formaldehyde on the activity of the sarcosine oxidase. Acetate and propionate at 0.1 g/l sarcosine are resulting in a substantial higher inhibition of the sarcosine oxidase compared to 1.0 g/l sarcosine, while the inhibition from acetaldehyde and formaldehyde is only slightly influenced by the substrate concentration. The measurements show the fact that also by using the stirred vessel system the amount of inhibition and therefore the selectivity of the reaction system can be influenced by variation of the substrate concentration.

Parallel Flow Analysis System (PFA System)

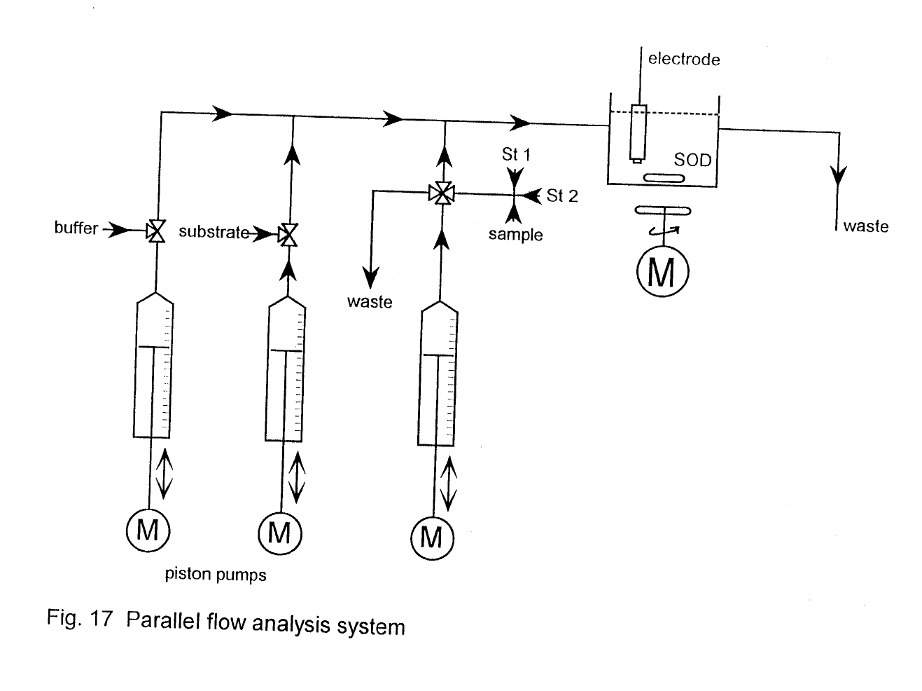

FIG. 17 shows the invented measuring device consisting of three piston pumps, which are pumping the respective buffer solution, the substrate and the sample containing the analyte or perhaps one of several possible calibration solutions (St). The piston pumps are connected to the reaction chamber via tubing and equiped with a valve, respectively. The reaction chamber contains the immobilized sarcosine oxidase. An oxygen or hydrogen peroxide electrode is used as a detector and it is fixed inside the reaction chamber.

The timing control of the three piston pumps is done in such a manner, that the probe which is mixed with substrate and buffer solution, is brought to a hold inside the reaction chamber. Increase of oxygen respectively decrease of hydrogen peroxide is measured as a timedepended value, where the measuring result is received from the initial gradient of the signal, similar to the stirred vessel system.

In order to calibrate via the initial inclines, samples of different analyte concentration are added and the appropriate initial gradients in dependence of the analyte concentration are drawn into the chart. The sample with unknown analyte concentration is brought in that same manner into the measuring chamber and determined there.

With the help of the PFA system a variation of the mixing proportions of buffer solution, substrate and sample are possible without any changes in apparatus simply by changing flow rate and suction volume of the appropriate piston pump. Especially this is necessary by the determination of samples of different matrix. Also an external dillution of the sample in order to precondition the sample can be renounced.

1. Method for the quantitative determination of reversible working inhibitors of the oxidoreductases, wherein said enzyme substrate is submitted and in presence of the inhibitor the enzyme activity of the enzyme, which is taking part in the reaction, is measured and by means of the data obtained the concentration of the inhibitor is determined.

2. The method of claim 1, wherein said sarcosine is used as substrate and said enzyme activity of the sarcosine oxidase is measured.

3. The method of claim 1 or 2, wherein said enzyme is used in immobilized state.

4. The method of claim 1 to 3, wherein said to increase the selectivity of the measurement the substrate, enzyme and buffer concentration, the buffer composition and substrate nature and/or the velocity of flow and buffer temperature is varied.

5. The method of claim 1 to 4, wherein said to increase the selectivity of the measurement by adding reversible working inhibitors of different kind and concentration to the reaction system.

6. The method of claim 1 to 5, wherein said the longtime loss of enzyme activity is compensated by an increase of substrate concentration.

7. Measuring device to perform the method of claim 1 to 6, wherein said at least two piston pumps are connected via feeding tubes to the reaction zone, which includes the enzyme, and said a detector which is situated inside the reaction zone or following it.

8. Measuring device of claim 7, wherein said in the feeding tubes are situated valves between the piston pumps and the reaction zone.

9. Measuring device of claim 7 or 8, wherein said detection of oxygen or hydrogen peroxide is done amperometrical or optical.

10. Test kit for the quantitative determination of reversible working inhibitors of the oxidoreductases comprising one oxidoreductase, a substrate of the oxidoreductase, a buffer and if necessary stabilizers.
